(19) United States
(12) Patent Application Publication
(10) Pub. No.: US 2009/0326583 A1
(43) Pub. Date: Dec. 31, 2009

(54) Posterior Dynamic Stabilization System With Flexible Ligament

(76) Inventors: Missoum Moumene, Newton, MA (US); Jonathan Fanger, Raynham, MA (US); Charles M Bartish, JR., Providence, RI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/145,714

(22) Filed: Jun. 25, 2008

(51) Int. Cl. A61B 17/70; A61B 17/04

(52) U.S. Cl. 606/257; 606/246; 606/264; 606/301

(57) ABSTRACT

A posterior dynamic spinal stabilization system having a sock or sleeve as the ligament to join a split rod so that during flexion, the ligament becomes taut to create an elongation limit, and during extreme extension, the upper and lower bumpers come together, thereby preventing further extension.

### BACKGROUND OF THE INVENTION

The vertebrae in a patient's spinal column are linked to one another by the disc and the facet joints, which control movement of the vertebrae relative to one another. Each vertebra has a pair of articulating surfaces located on the left side, and a pair of articulating surfaces located on the right side, and each pair includes a superior articular surface, which faces upward, and an inferior articular surface, which faces downward. Together the superior and inferior articular surfaces of adjacent vertebra form a facet joint. Facet joints are synovial joints, which means that each joint is surrounded by a capsule of connective tissue and produces a fluid to nourish and lubricate the joint. The joint surfaces are coated with cartilage allowing the joints to move or articulate relative to one another.

Diseased, degenerated, impaired, or otherwise painful facet joints and/or discs can require surgery to restore function to the three joint complex. Damaged, diseased levels in the spine were traditionally fused to one another. While such a technique may relieve pain, it effectively prevents motion between at least two vertebrae. As a result, additional stress may be applied to the adjoining levels, thereby potentially leading to further damage.

More recently, techniques have been developed to restore normal function to the facet joints. One such technique involves covering the facet joint with a cap to preserve the bony and articular structure. Capping techniques, however, are limited in use as they will not remove the source of the pain in osteoarthritic joints. Caps are also disadvantageous as they must be available in a variety of sizes and shapes to accommodate the wide variability in the anatomical morphology of the facets. Caps also have a tendency to loosen over time, potentially resulting in additional damage to the joint and/or the bone support structure containing the cap.

Other techniques for restoring the normal function to the posterior element involve arch replacement, in which superior and inferior prosthetic arches are implanted to extend across the vertebra typically between the spinous process. The arches can articulate relative to one another to replace the articulating function of the facet joints. One drawback of current articulating facet replacement devices, however, is that they require the facet joints to be resected. Moreover, alignment of the articulating surfaces with one another can be challenging.

Accordingly, there remains a need for improved systems and methods that are adapted to mimic the natural function of the facet joints.

US Patent Publication 2004-0225289 (Biedermann I) discloses a dynamic anchoring device is described. An element with a shank for anchoring in a bone or a vertebra and with a head connected to the shank is provided with a receiving part for the head and with an elastomeric pressure element acting on the head. The pressure element is formed and located in such a way that, upon a movement of the element from a first angular position of the shank relative to the receiving part into a second angular position, it exerts a return force on the head. Further, a dynamic stabilization device, in particular for vertebrae, is provided. In such a stabilization device, a rod is connected two anchoring devices. At least one of the anchoring devices is constructed as dynamic anchoring element

US Patent Publication 2005-0154390 (Biedermann II) discloses an elastic or flexible element for use in a stabilization device for bones or vertebrae. The elastic or flexible element is provided in the form of an essentially cylindrical body with a first end and a second end opposite thereto, wherein at least one of the opposite ends of the cylindrical body comprises a coaxial bore hole with an internal thread for connecting to a shaft and/or a head of a bone screw or for connecting to a rod section. The present invention further provides a bone anchoring element, e.g. a bone screw, with a shaft for the anchoring in a bone, whereby the shaft comprises an elastic or flexible section which is formed integrally with the shaft or as a separate elastic or flexible element. It is preferable for the elastic section to be implemented in the form of a helical spring. Moreover, the present invention provides a stabilization device for bones, for instance for vertebrae, said device comprising at least one bone anchoring element according to the invention, a second bone anchoring element and a rod or plate connecting the bone anchoring elements.

EP Patent Publication 1579816 (Biedermann III) discloses an anchoring element comprises a receiving part connected to a shaft for receiving a rod-shaped element, and a fixation device for fixing the rod-shaped element in the receiving part. It also discloses an anchoring element comprises a receiving part connected to the shaft for receiving the rod-shaped element, and a fixation device for fixing the rod-shaped element in the receiving part, where the shaft is connected by the receiving part to the rod-shaped element in a mobile fashion so that the shaft can move with respect to the rod-shaped element with at least one degree of rotational freedom, but no degree of translational freedom in the fixed state.

US Patent Publication 2005-0143823 (Boyd) discloses a dynamic stabilization construct for implantation within the spine comprises bone anchors that include a flexible portion between the bone engaging and head portions of the anchor. The head portion is configured to mate with different types of stabilization elements adapted to span between spinal motion segments. The engagement portion can also be configured for different types of fixation to a motion segment, such as within the pedicle of a vertebra. The flexible portion permits limited bending of the bone anchor beneath the level of the stabilization element. In one embodiment, the flexible portion is integrated into the body of the bone anchor in the form of hinge elements. In another embodiment, a separate flexible element, such as a spacer or spring, is interposed between the head and engagement portions. In a further embodiment, the bone anchor includes a portion having a reduced cross-section. The flexible bone anchors may be used to tailor the dynamic flexibility of spinal stabilization instrumentation at each level of the construct

US Patent Publication 2005-0182409 (Callahan) discloses a motion interface structure for use with a pedicle screw is provided, the motion interface structure defining a central passage having an internal face. A helical thread is formed on at least a portion of the internal face of the central passage. The motion interface element is designed to cooperate with an upstanding region of a pedicle screw. The upstanding region includes a threaded region that is adapted to threadingly engage the helical thread associated with the motion interface element. The motion interface element may take the form of a spherical element or a universal joint mechanism. The pedicle screw and motion interface element may be incorporated into a spinal stabilization system that includes one or more additional pedicle screw/motion interface element subassemblies. The spinal stabilization system may also include a dynamic stabilizing element that provides clinically efficacious results.

US Patent Publications 2004-0236329 (Panjabi) and 2005-0222659 (Panjabi II) discloses a dynamic spine stabilizer moves under the control of spinal motion providing increased mechanical support within a central zone corresponding substantially to the neutral zone of the injured spine. The dynamic spine stabilizer includes a support assembly and a resistance assembly associated with the support assembly. The resistance assembly generates greater increase in mechanical force during movement within the central zone and lesser increase in mechanical force during movement beyond the central zone. A method for using the stabilizer is also disclosed.

US Patent Publications 2004-0236327 (Paul I) and 2004-0236328 (Paul II) disclose a spine stabilization system having one or more flexible elements with tubular structures with openings or slits. The flexible elements may limit rotation, flexion-extension, or lateral bending of the spine. The system also may have a locking mechanism that secures one or more flexible elements in a rigid configuration. A flexible element may be disposed within another flexible element, and the slits may form helical patterns on the tubular structures. The flexible element may be conformable to the natural spinal movement.

US Patent Publication 2005-0171543 (Timm I) discloses a system and method for effecting multi-level spine stabilization. The system includes a plurality of pedicle screws which are joined relative to each other by elongated members, e.g., rods. At least one of the rods includes a dynamic stabilizing member. The pedicle screw junctions are dynamic, i.e., free relative movement of a socket member is permitted relative to a fixed spherical element. Placement of the spherical element may be facilitated using a guidewire system that includes a guidewire and a tapered guide member. A spine stabilization assembly is also provided that includes an attachment member that includes an opening. At least one spherical element that includes a rod-receiving channel is movably mounted within the opening with three degrees of rotational freedom. The spherical element generally defines an elliptical rod-receiving channel that is deformable to a circular opening to firmly engage a rod positioned therein. Multi-level stabilization systems that combine/mix dynamic and non-dynamic stabilization modalities are also provided. The multi-level spine stabilization system offers efficacious clinical results at least in part due to the inclusion of dynamic stabilizing member(s).

US Patent Publication 2005-0182401 (Timm II) discloses a spinal stabilization devices, systems and methods are provided that include at least one pedicle screw and at least one mechanism that supports three degrees of rotational freedom relative to the pedicle screw. The mechanism may include a universal joint mechanism or a ball and socket mechanism. In the case of the ball and socket mechanism, at least one spherical element is mounted with respect to the at least one pedicle screw and a socket member cooperates with the spherical element. The spherical element and the socket member cooperate to define a dynamic junction that allows the socket member to move relative to the ball element while remaining engaged therewith. The dynamic junction is advantageously incorporated into a spinal stabilization system that includes additional pedicle screw(s), spherical element(s) and socket member(s). The spinal stabilization system may incorporate dynamic stabilizing member(s) to so as to provide clinically efficacious results

US Patent Publication 2005-0177164 (Walters) discloses a pedicle screw assembly that includes a pedicle screw and a preloaded set screw. The set screw is preloaded in a threaded, central aperture formed in the head region of the pedicle screw. An interference is advantageously formed on the set screw to prevent dislodgement of the set screw, e.g., during shipment and/or clinical placement of the pedicle screw. An upwardly extending collet is generally formed in the head region of the pedicle screw, the collet being sized to receive a spherical element therearound. Advancement of the set screw relative to the pedicle screw secures the spherical element relative to the pedicle screw. The spherical element typically includes a socket member that cooperates with a dynamic stabilizing member. The pedicle screw assembly and dynamic stabilizing member are advantageously used as part of a spinal stabilization system to provide clinically efficacious results.

US Patent Publication 2005-0182400 (White) discloses a system and method for facilitating a spinal stabilization procedure. A tapered guide member is positioned adjacent to or in juxtaposition with the head of a pedicle screw, and the associated components are thus guided into alignment therewith. A component, e.g., a spherical element, may be advanced onto a collet that extends upwardly from the head of the pedicle screw. A guidewire may also be employed to guide components to the pedicle screw and/or to guide the guidewire into position. Thus, a conical guide member may be slid down a guidewire into alignment with a pedicle screw, and subsequently advanced components may be guided into alignment with the pedicle screw. The tapered guide member may include registration feature(s) and may facilitate alignment with off-axis locations. The facilitating system may be employed with a dynamic spinal stabilization system that provides clinically efficacious results at least in part based upon inclusion of dynamic stabilizing member(s).

### SUMMARY OF THE INVENTION

In accordance with the present invention, there is provided a posterior dynamic spinal stabilization system for use in a human spine, comprising:
a) first and second bone anchors, each anchor having a recess for receiving a rod,
b) first and second rod portions, each rod portion having an outer end portion received in the recess of the bone anchor and an inner end portion having an inner end face,
c) a ligament having a first end portion and a second end portion,
   wherein the outer end portion of the first rod portion is received in the recess of the first bone anchor,
   wherein the outer end portion of the second rod portion is received in the recess of the second bone anchor,
   wherein the inner end faces of the rod portions oppose each other, and
   wherein the first end portion of the ligament is attached to the inner end portion of the first rod portion, and the second end portion of the ligament is attached to the inner end portion of the second rod portion.

Preferably, this invention uses a sock or sleeve as the ligament to join the two elastomeric inner end faces, or bumpers. During extreme flexion, the ligament becomes taut to create an elongation limit. During extreme extension, the upper and lower bumpers contact each other, thereby preventing further extension.

The present invention can limit undesirable excessive motion by way of an elastomer or woven polymer ligament that changes shape to allow some flexion motion. With progressive flexion, the weave becomes tighter or looser and the elastomer stretches to restrict further flexion.

The present invention can limit flexion by providing a ligament (or sleeve) that has slack. The sleeve functions as an elongation stop which does not provide any stiffness in flexion.

The present invention may also limit shear and some torsion by means of a piston disposed between the adjacent bumpers.

### DESCRIPTION OF THE FIGURES

FIG. 1 discloses the device of the present invention during extension of the functional spinal unit.

FIG. 2 discloses the device of the present invention during flexion of the functional spinal unit.

FIG. 3 discloses the device of the present invention having a piston.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

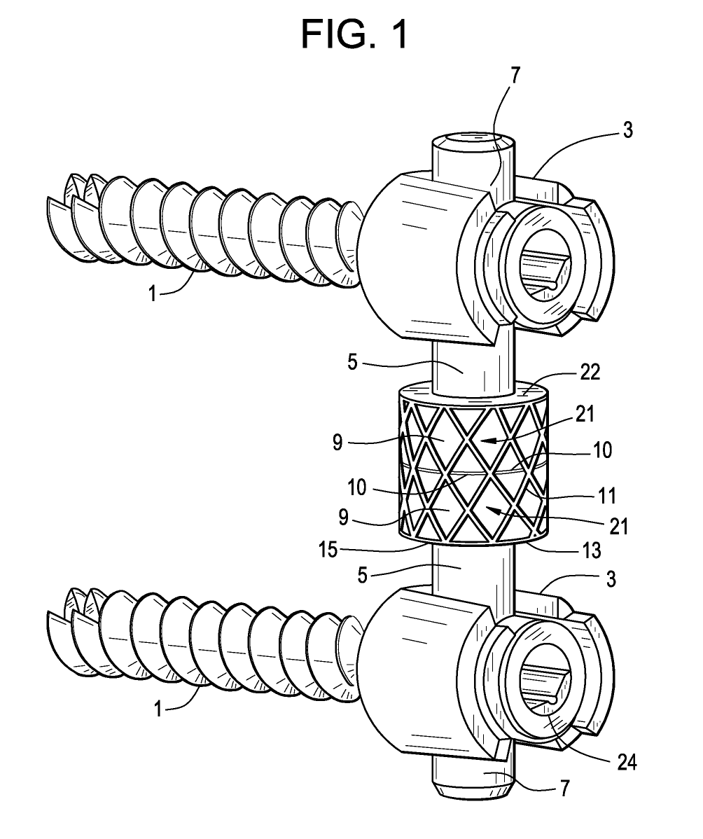

Now referring to FIG. 1, there is provided a posterior dynamic spinal stabilization system, comprising:
a) first and second bone anchors 1, each anchor having a recess 3 for receiving a rod,
b) first and second rod portions 5, each rod portion having an outer end portion 7 received in the recess of the bone anchor and an inner end portion 9 (preferably, comprising a bumper) having an inner end face 10,
c) a ligament 11 having a first end portion 13 and a second end portion 15,
   wherein the outer end portion of the first rod portion is received in the recess of the first bone anchor,
   wherein the outer end portion of the second rod portion is received in the recess of the second bone anchor,
   wherein the inner end faces of the rod portions oppose each other, and
   wherein the first end portion of the ligament is attached to the inner end portion of the first rod portion, and the second end portion of the ligament is attached to the inner end portion of the second rod portion.

FIG. 1 discloses the device of the present invention during extension of the functional spinal unit. As shown, a traditional pedicle screw may be used in accordance with this embodiment. A rod comprising first and second rod portions is assembled to the dynamic ligament, enabling attachment to the pedicle screws. The rod could be made of any biocompatible plastic or metallic material, while the bumper is preferably made of an elastomeric material capable of acting as an extension stop.

Therefore, also in accordance with the present invention, there is provided a posterior dynamic spinal stabilization system, comprising:
a) first and second bone anchors, each anchor having a recess for receiving a rod,
b) first and second rod portions, each rod portion having an outer end portion received in the recess of the bone anchor and an inner end portion having an inner end face,
   wherein the outer end, portion of the first rod portion is received in the recess of the first bone anchor,
   wherein the outer end portion of the second rod portion is received in the recess of the second bone anchor,
   wherein the inner end faces of the rods oppose each other, and
   wherein the outer end portion of each rod portion comprises a plastic or metallic material, and the inner end portion of each rod portion comprises an elastic material.

The ligament is preferably present in the form of a dynamic tubular sock component that acts as a sleeve joining the two bumpers. The sock component is able to elongate during functional spinal unit flexion. The sock or sleeve could be made from an inelastic polymer, such as a braided or woven suture material, which would simply provide an elongation stop as the ligament becomes taut. Non-elastic ligament materials would likely achieve elongation by increasing the tightness of the weave as the rod extends. The ligament could also be made from an elastomeric material that stretches during elongation. A number of other suitable materials could be used as long as they were biocompatible and accomplished the intent of the device.

In some embodiments, the inner end portion of each rod portion has a diameter greater than the diameter of the outer end portion of each rod portion, as in FIG. 1. In this condition, the inner end faces have a greater surface area, and so more evenly distribute contact stresses produced during extension.

In some embodiments, the inner end portion of each rod portion has a peripheral surface 21, and the ligament is attached to the peripheral surface of each inner end portion, as in FIG. 1. Attachment to the peripheral surface allows a greater attachment area for a tubular ligament, and so reduces the tension placed upon the ligament during its elongation in response to flexion.

In some embodiments, the inner end portion of each rod portion forms a ledge 22, and the ligament is attached to the ledge.

In some embodiments, the ligament is attached to both the peripheral surface and ledge of each inner end portion.

In some embodiments, the ligament is tubular and is circumferentially attached to the peripheral surface of each inner end portion of each rod portion, as in FIG. 1. Circumferential attachment to the peripheral surface provides a maximum attachment area for a tubular ligament, and so minimizes the tension placed upon the ligament during its elongation in response to flexion.

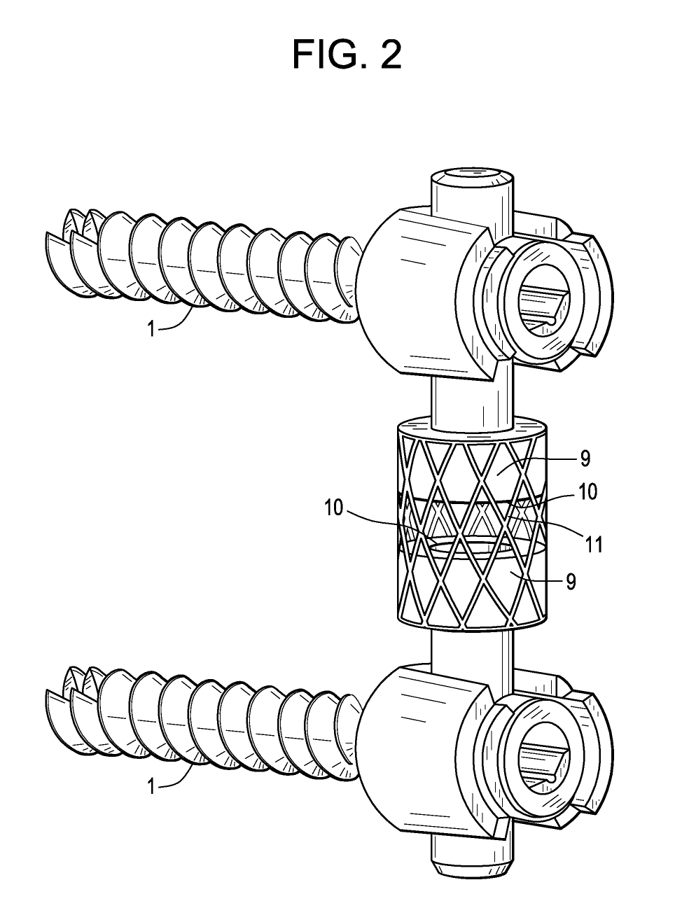

FIG. 2 discloses the device of the present invention during flexion of the functional spinal. The sock component 11 (shown as extended in FIG. 2) would have this elongated shape during functional spinal unit flexion. The elastomeric bumpers 9 and sock 11 form the dynamic components of this device.

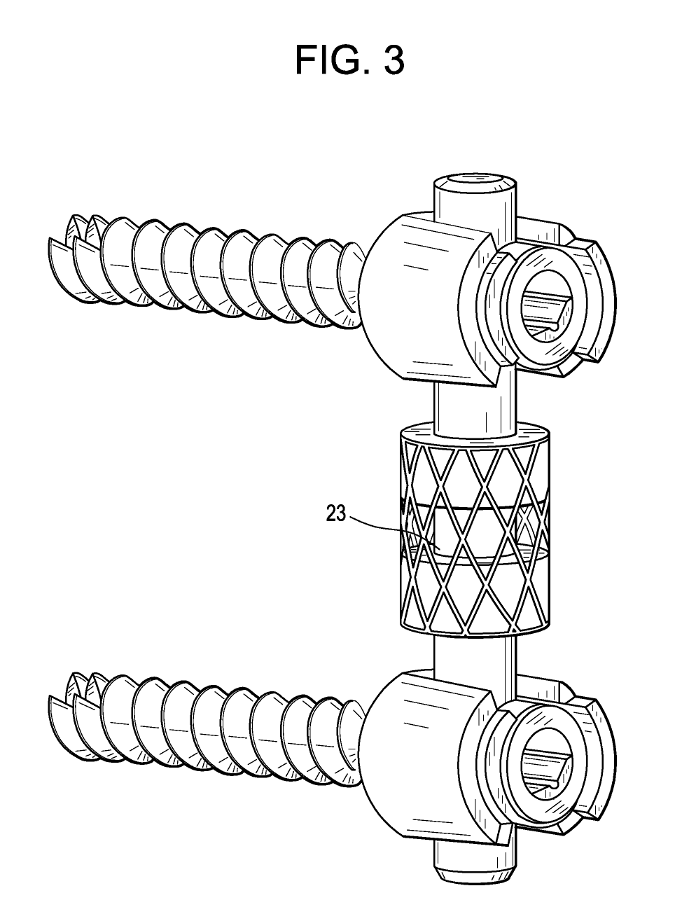

Now referring to FIG. 3, in some embodiments, the system further comprises: d) a piston 23 having a first annulus disposed on the first inner end face and a second annulus disposed on the second inner end face, wherein the first annulus is slidably received in the second annulus. To further improve shear and torsional resistance of the device, the piston may be present between the inner end faces of the two rod portions.

In each of these designs, the geometry of the bumpers can be altered to better control tension within the sock. The bumpers may be supplied in conical, radiused, tapered, or other shapes that create more favorable loading within the sock.

In general, the bone anchors are made from metallic materials; the rod can be made from metallic, ceramic or polymeric materials; and the ligament is made of polymeric materials or more preferably, elastomeric materials.

In some embodiments, the ligament is inelastic and is preferably braided or woven. In other embodiments, the ligament is elastic.

If a metal is chosen as the material of construction, then the metal is preferably selected from the group consisting of nitinol, titanium, titanium alloys (such as Ti-6Al-4V), chrome alloys (such as CrCo or Cr—Co—Mo) and stainless steel.

If a polymer is chosen as a material of construction, then the polymer is preferably selected from the group consisting of polycarbonates, polyesters, (particularly aromatic esters such as polyalkylene terephthalates, polyamides; polyalkenes; poly(vinyl fluoride); PTFE; polyarylethyl ketone PAEK; and mixtures thereof.

In some embodiments, the bone anchors are made of a stainless steel alloy, preferably BioDur^{R} CCM Plus^{R} Alloy available from Carpenter Specialty Alloys, Carpenter Technology Corporation of Wyomissing, Pa. In some embodiments, the rod is made from a composite comprising carbon fiber. Composites comprising carbon fiber are advantageous in that they typically have a strength and stiffness that is superior to neat polymer materials such as a polyarylethyl ketone PAEK. In some embodiments, the tube is made from a polymer composite such as a PEKK-carbon fiber composite.

Preferably, the composite comprising carbon fiber further comprises a polymer. Preferably, the polymer is a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the rod is made from a neat polymer without any carbon fiber additive. Preferably, the polymer is a polyarylethyl ketone (PAEK), more preferably PEEK.

In some embodiments, the carbon fiber comprises between 1 vol % and 60 vol % (more preferably, between 10 vol % and 50 vol %) of the composite. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite comprises:
a) 40-99% (more preferably, 60-80 vol %) polyarylethyl ketone (PAEK), and
b) 1-60% (more preferably, 20-40 vol %) carbon fiber,
   wherein the polyarylethyl ketone (PAEK) is selected from the group consisting of polyetherether ketone (PEEK), polyether ketone ketone (PEKK) and polyether ketone (PEK).

In some embodiments, the composite consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

The elastomeric ligament can preferably be formed from polycarbonate, but may also be formed of any other elastomeric biocompatible material depending on the properties desired. Generally, the elastomeric ligament is made of an elastomer, and may be preferably an elastomer as selected in U.S. Pat. No. 5,824,094 (“Serhan”). In some embodiments, the elastomeric ligament is preferably made of a polyolefin rubber or carbon black reinforced polyolefin rubber. The hardness of the elastomeric ligament may be preferably 56-72 shore A durometer. The ultimate tensile strength of the ligament may be preferably greater than 1600 psi. The ligament may have an ultimate elongation greater than 300% using the ASTM D412-87 testing method, and a tear resistance greater than 100 psi using the ASTM D624-86 testing method. Although the elastomeric ligament is disclosed as being made of a polyolefin rubber or polycarbonate in some embodiments, it can be made of any elastomeric material that simulates the characteristics of natural ligaments. In some embodiments, the ligament is made of UHMWPE.

One skilled in the art will appreciate that the rod of the device may be configured for use with any type of bone anchor, e.g., bone screw or hook; mono-axial or polyaxial. Typically, a bone anchor assembly includes a bone screw, such as a pedicle screw, having a proximal head and a distal bone-engaging portion, which may be an externally threaded screw shank. The bone screw assembly may also have a receiving member that is configured to receive and couple a spinal fixation element, such as a spinal rod or spinal plate, to the bone anchor assembly.

In some embodiments, the bone anchor has a plate and bolt design.

The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, the specification of which is incorporated herein by reference in its entirety. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, the specifications of which are incorporated herein by reference in their entireties.

Generally, in using the present invention, two bone anchors such as polyaxial screws are inserted into adjacent pedicles within a functional spinal unit of a patient. The rod-ligament assembly of the present invention is then inserted into the patient between the anchors. The outer end portion of the first rod portion of the rod-ligament assembly is attached to the first bone anchor by laying the outer end portion of the first rod portion into the first bone anchor recess and tightening the appropriate set screw 24. Similarly, the outer end portion of the second rod portion of the rod-ligament assembly is attached to the second bone anchor by laying the outer end portion of the second rod portion into the second bone anchor recess and tightening the appropriate set screw 24 (in FIG. 1). More preferably, this is achieved in a minimally invasive surgery.

Therefore, in accordance with the present invention, there is provided a method of implanting a posterior dynamic spinal stabilization system, comprising the steps of:
a) inserting two bone anchors into adjacent pedicles within a functional spinal unit of a patient, each bone anchor having a recess for receiving a rod,
b) providing rod-ligament assembly comprising:
   i) first and second rod portions, each rod portion having an outer end portion received in the recess of the bone anchor and an inner end portion having an inner end face,
   ii) a ligament having a first end portion and a second end portion,
   wherein the inner end faces of the rod portions oppose each other, and
      wherein the first end portion of the ligament is attached to the inner end portion of the first rod portion, and the second end portion of the ligament is attached to the inner end portion of the second rod portion,
c) fastening the outer end portion of each rod portion into the respective bone anchor recess.

In addition, the present invention can be used with a multi-level rod. In some embodiments thereof, there is provided a three-anchor construct having a central rod for the center bone screw having an end extending from each side. The three-anchor construct includes:
a) at least three bone anchors adapted for receiving a rod;
b) a rod comprising:
   i) first and second outer rod portions, each having an outer end portion received in the recess of the bone anchor and an inner end portion having an inner end face,
   ii) an intermediate rod portion having a middle portion received in the recess of the bone anchor and two outer portions having an outer end face extending from each end of the intermediate rod portion, and
c) a ligament having a first end portion and a second end portion.
   wherein the intermediate rod portion is disposed between the first and second outer rod portions, so that the outer end faces of the intermediate portion face the inner end faces of the outer rod portions, and
   wherein the first end portion of the ligament is attached to the first outer rod portion, and wherein the second end portion of the ligament is attached to the second outer rod portion.

In addition, the rods of the present invention can include any suitable cross-section, including non-circular cross sections.

We claim:

1. A posterior dynamic spinal stabilization system, comprising:
a) first and second bone anchors, each anchor having a recess for receiving a rod,
b) first and second rod portions, each rod portion having an outer end portion received in the recess of the bone anchor and an inner end portion having an inner end face,
c) a ligament having a first end portion and a second end portion,
wherein the outer end portion of the first rod portion is received in the recess of the first bone anchor,
wherein the outer end portion of the second rod portion is received in the recess of the second bone anchor,
wherein the inner end faces of the rod portions oppose each other, and
wherein the first end portion of the ligament is attached to the inner end portion of the first rod portion, and the second end portion of the ligament is attached to the inner end portion of the second rod portion.

2. The system of claim 1 wherein the bone anchor is a screw.

3. The system of claim 1 wherein the bone anchor is a polyaxial screw.

4. The system of claim 1 wherein the outer end portion of each rod portion comprises a plastic or metallic material, and the inner end portion of each rod portion comprises an elastic material.

5. The system of claim 1 wherein the inner end portion of each rod portion has a peripheral surface, and the ligament is attached to the peripheral surface of each inner end portion.

6. The system of claim 5 wherein the ligament is tubular and is circumferentially attached to the peripheral surface of each inner end portion of each rod portion.

7. The system of claim 6 wherein the ligament is inelastic.

8. The system of claim 7 wherein the inelastic ligament is braided or woven.

9. The system of claim 6 wherein the ligament is elastic.

10. The system of claim 1 wherein the inner end portion of each rod portion forms a ledge, and the ligament is attached to the ledge.

11. The system of claim 10, wherein the inner end portion of each rod portion has a peripheral surface, wherein the ligament is attached to both the peripheral surface and ledge of each inner end portion

12. The system of claim 1 further comprising:
d) a piston having a first annulus disposed on the first inner end face and a second annulus disposed on the second inner end face, wherein the first annulus is slidably received in the second annulus..

13. A method of implanting a posterior dynamic spinal stabilization system, comprising the steps of:
a) inserting two bone anchors into adjacent pedicles within a functional spinal unit of a patient, each bone anchor having a recess for receiving a rod,
b) providing a rod-ligament assembly comprising:
i) first and second rod portions, each rod portion having an outer end portion received in the recess of the bone anchor and an inner end portion having an inner end face,
ii) a ligament having a first end portion and a second end portion,
wherein the inner end faces of the rod portions oppose each other, and
wherein the first end portion of the ligament is attached to the inner end portion of the first rod portion, and the second end portion of the ligament is attached to the inner end portion of the second rod portion,
c) fastening the outer end portion of each rod portion into the respective bone anchor recess.

14. The method of claim 13 wherein the fastening step includes laying the outer end portion of each rod portion into the respective bone anchor recess and tightening with a set screw thereon.

15. A posterior dynamic spinal stabilization system, comprising:
a) first and second bone anchors, each anchor having a recess for receiving a rod,
b) first and second rod portions, each rod portion having an outer end portion received in the recess of the bone anchor and an inner end portion having an inner end face,
wherein the outer end portion of the first rod portion is received in the recess of the first bone anchor,
wherein the outer end portion of the second rod portion is received in the recess of the second bone anchor,
wherein the inner end faces of the rod portions oppose each other, and
wherein the outer end portion of each rod portion comprises a polymeric or metallic material, and the inner end portion of each rod portion comprises an elastic material.

16. The system of claim 15 wherein the bone anchor is a polyaxial screw.

17. The system of claim 15 wherein the inner end portion of each rod portion has a peripheral surface, and the ligament is attached to the peripheral surface of each inner end portion.

18. The system of claim 17 wherein the ligament is tubular and is circumferentially attached to the peripheral surface of each inner end portion of each rod portion.

19. The system of claim 15 wherein the inner end portion of each rod portion forms a ledge, and the ligament is attached to the ledge.

20. The system of claim 19, wherein the inner end portion of each rod portion has a peripheral surface, wherein the ligament is attached to both the peripheral surface and ledge of each inner end portion

21. The system of claim 15 wherein the ligament is inelastic.

22. The system of claim 21 wherein the inelastic ligament is braided or woven.

23. The system of claim 15 wherein the ligament is elastic.

24. The system of claim 15 further comprising:
d) a piston having a first annulus disposed on the first inner end face and a second annulus disposed on the second inner end face, wherein the first annulus is slidably received in the second annulus.

25. The system of claim 15 wherein the inner end portion of each rod is conical.

26. The system of claim 15 wherein the inner end portion of each rod is radiused.

27. The system of claim 15 wherein the inner end portion of each rod is tapered.

28. The system of claim 15 wherein the inner end portion of each rod has a diameter greater than a diameter of the outer end portion of each rod portion.

29. A posterior dynamic spinal stabilization system, comprising:
a) at least three bone anchors adapted for receiving a rod;
b) a rod comprising:
i) first and second outer rod portions, each having an outer end portion received in the recess of the bone anchor and an inner end portion having an inner end face,
ii) an intermediate rod portion having a middle portion received in the recess of the bone anchor and two outer portions having an outer end face extending from each end of the intermediate rod portion, and
c) a ligament having a first end portion and a second end portion.
wherein the intermediate rod portion is disposed between the first and second outer rod portions, so that the outer end faces of the intermediate portion face the inner end faces of the outer rod portions, and
wherein the first end portion of the ligament is attached to the first outer rod portion, and wherein the second end portion of the ligament is attached to the second outer rod portion.
